(19) United States
(12) Patent Application Publication
(10) Pub. No.: US 2017/0119553 A1
(43) Pub. Date: May 4, 2017

(54) A HAPTIC FEEDBACK DEVICE

(71) Applicants: Scuola Superiore S.Anna, Pisa (IT); Uminova Holding AB, Umeå (SE)

(72) Inventors: Christian CIPRIANI, Lucca (IT); Francesco CLEMENTE, Sant'elia Fiumerapido (IT); Marco D'ALONZO, Pontedera (IT); Benoni B. EDIN, Umeå (SE)

(73) Assignees: Scuola Superiore S.Anna, Pisa (IT); Uminova Holding AB, Umeå (SE)

(21) Appl. No.: 15/320,224

(22) PCT Filed: Jun. 19, 2015
(86) PCT No.: PCT/IB2015/054639

§ 371 Date: Dec. 19, 2016

(30) Foreign Application Priority Data
Jun. 20, 2014 (IT) PI2014A000045

(51) Int. Cl. A61F 2/76; A61F 2/68

(52) U.S. Cl. CPC A61F 2/76; A61F 2/68; A61F 2002/6827; A61F 2002/7635; A61F 2002/7665; A61F 2002/764

(57) ABSTRACT

A haptic feedback device (10) for a prosthetic body part (2) or a sensorially deficient body part (2′) comprises a sensor interface (12) configured to be arranged on the prosthetic or sensorially deficient body part (2,2′), equipped with a contact sensor (21) to generate a contact signal (23) according to a contact condition with an object (6); a tactile stimulation unit equipped with a transducer (13,13′) to generate a tactile stimulation (33); a support (15) wearable on a sensorially sound body part (5), on which the tactile stimulation unit is arranged, so as to receive the stimulation (33); a control unit (14) for receiving the contact signal (23) from the contact sensor (21) and comprising a program means (41) configured to cause a control signal (29) to be produced for actuating the transducer, responsive to the contact signal (23), and for cyclically comparing the contact signal (23) with a contact threshold value (Ti,j); for producing the control signal (29) and actuating the transducer (13,13′) when the contact signal (23,23′) crosses the threshold value (Ti,j); for leaving the transducer (13,13′) still in an intermediate time between this crossing of the threshold (Ti,j) and a subsequent crossing in an opposite direction, so that the transducer (13) is actuated if an event occurs of beginning or of end of a contact between the prosthetic or sensorially deficient body part (2,2′) and the object (6).

### FIELD OF THE INVENTION

The present invention relates to a haptic device for providing a haptic feedback to a user of a prosthetic a body part, or to a user who has a sensorially deficient body part, for example due to an ictus, or to a spinal column trauma, and so on.

More in detail, the invention relates to a device for providing a haptic feedback to the user, in connection to an interaction with an external object, such as contacting, gripping, or displacing this object.

### STATE OF THE ART

The loss of a limb causes serious functional limitations, typically in the ability of handling objects and of walking. These limitations are often partially compensated for by myoelectric prosthesis, which can more and more closely approximate the natural limb movements.

Myoelectric prosthesis are controlled by a closed loop technique that normally exploits a visual feedback, which allows the user, for example, to check whether a gripping process is being correctly performed or not. As an alternative, secondary feedbacks are used as they are provided, for instance, by the vibrations and by the noise of the prosthesis.

However, it is known that a tactile feedback would improve the prosthesis closed loop control procedure, besides providing data concerning the outer environment. To this purpose, two approaches are possible, which are useful also for non-amputees who have a sensorially deficient part of their own body:

feedback by sensorial stimulation similitude: it consists in applying or “transferring” in a distinct body part a stimulation similar to the one that acts on the prosthesis or on the sensorially deficient member. For instance, the pressure acting on a fingertip can be turned into a pressure applied somewhere else in the body. However, it is difficult to provide actuators that have suitably small size, weight and energy requirements, moreover the available neural interfaces have a low selectivity and a short duration, which makes this approach unsuccessful.

feedback by sensorial stimulation substitution: it consists in providing a tactile information by means of a stimulation of a different type with respect to the original one, for instance, by replacing a pressure acting on the skin, by vibrations applied on sensorially sound body parts. Actuators are available which are suitable for this purpose, and which have a small weight, size, energy consumption and cost. This leaded to prefer this technique, to which many publications refer, such as EP1028675B1 and US20090048539.

However, the above devices the drawback of accustoming the user to a continuous stimulation during the contact steps, or in any case when using the prosthesis. For instance, the mechanical events of starting and stopping a sequence of manipulation are provided to the user as a modification of a tactile stimulation that is different from a pressure stimulation. The user must therefore make a brain effort to recognize the intensity change of these tactile stimulations. This complicates the recognition of the events and can tire the patient.

Furthermore, most prior art haptic feedback devices consume a large amount of energy to generate the tactile stimulations. To ensure a certain autonomy relatively heavy and bulky batteries are therefore required, which cause discomfort to the patient.

WO 98/25552 describes an apparatus to provide a subject with stimulations corresponding to an external operation on a sensor of a prosthesis, used in combination with a prosthetic limb or with a limb provided with sensors. A prosthetic lower/upper limb comprises sensors arranged in a prosthetic foot/thumb, vibrating contacts/a motor for producing stimulations to the stump, and an electronic for adjusting and controlling the intensity of the stimulations/the motor operation. An apparatus for a sensorially deficient limb is made similarly.

### SUMMARY OF THE INVENTION

It is therefore a feature of the invention to provide a haptic feedback device for a prosthesis bearer or for a user having a sensorially deficient body part, typically a limb, which does not require the user's adaptation to a stimulation, as it is the case for the prior art haptic feedback devices.

It is a particular feature of the invention to provide such a device that allows the user to recognize the contact of a prosthetic or sensorially deficient limb with an object, without requiring an excessive brain effort, and so allows him/her to recognize movements of the prosthesis or of the limb and/or displacements of objects caused by the prosthesis or by the limb.

It is another feature of the invention to provide such a device that consumes less energy than the prior art devices, and that requires therefore smaller batteries to provide a certain autonomy.

It is a particular feature of the invention to provide such a device that is lighter and smaller than the prior art devices, and that can be comfortably born by the user.

These and other objects are achieved by a haptic feedback device for a user's prosthetic or sensorially deficient body part, the device comprising:

a sensor interface configured to be arranged on a prosthetic or sensorially deficient body part,

wherein the sensor interface comprises a contact sensor configured to generate a contact signal according to a contact condition occurring between the sensor and an object of the outside of the user's body;

a tactile stimulation unit comprising a transducer configured to generate a tactile stimulation adapted to be perceived by a healthy user's body part;

a support configured to be worn on a region of the user's body, wherein the tactile stimulation unit is arranged on the support so as to transfer the tactile stimulation to the healthy body part;

a control unit configured to receive the contact signal from the sensor and to produce a control signal for actuating the transducer, wherein the control unit comprises a program means configured to cause the control unit to produce the control signal responsive to the contact signal, such that the transducer provides the tactile stimulation to the healthy body part;

wherein the program means is also configured to carry out steps of:

cyclically comparing the contact signal and a first contact threshold value corresponding to said contact condition;

producing the control signal and actuating the transducer when the contact signal carries out a first crossing of the first contact threshold value, such that the tactile stimulation unit transfers the tactile stimulation to the healthy body part;

leaving the transducer substantially still in an intermediate time between the first crossing of the first contact threshold value and a second crossing of a second contact threshold value by the contact signal, in a direction opposite to said first crossing,

so that the transducer is actuated at the beginning and/or at the end of a contact between the prosthetic or sensorially deficient body part and the object.

With the device according to the invention, the user receives an impulse tactile stimulation in the time, i.e. a tactile stimulation at the beginning or at the end of a condition of contact with the object. After this event, the control signal produced starting from the contact signal is discontinued, along with the tactile stimulation given to the patient, which specifically relate to such contact condition. In particular, during the time between the beginning and the end of the contact, the control unit does not transfer the transducers any control signal produced starting from a specific contact signal.

Therefore, it will be less difficult for the user to recognize the stimulations that correspond to the beginning and/or to the end of the contact, so the user will have to make a lighter mental effort less than in the case of the prior art devices, in order to recognize such events.

Moreover, by supplying a same amount of energy to the transducers, the device according to the invention is able to notify a much larger number of events than the prior art devices. In fact, the transducer of the device according to the invention consumes energy substantially only while generating the tactile stimulations, which are practically instantaneous.

For this reason, in a same operation time, much smaller and lighter batteries are required than in the prior art devices. Therefore, the device according to the invention can be more comfortably worn than most prior art devices.

In particular, the transducer can be selected among the transducers configured to generate mechanical tactile stimulations, more in particular, tactile stimulations comprising a plurality of mechanical vibrations, electric stimulations, or thermal stimulations.

By contact sensor a device is meant that is configured to receive an input due to the contact with an external body, to measure an own variable property by means of this contact, and to provide a contact signal according to this property. The signal can be an impulse detection signal having a predetermined value, in particular an impulse signal generated when at least one threshold value of this feature is exceeded. As an alternative, the signal can be can be a measurement signal continuously depending on the value of the feature that changes responsive to the contact.

In an exemplary embodiment, the contact sensor can be a force sensor, i.e. a device configured to receive a force due to the contact with the object. In alternative exemplary embodiments, the contact sensor can be a thermal sensor, i.e. a temperature sensor, in which case the input is a heat transfer caused by a temperature difference between the sensor and the external body, an electromagnetic sensor, in particular an optical sensor, in which case the input is an energy transfer associated with an electromagnetic field, a acoustic sensor, in particular an ultrasound sensor, in which case the input is an energy transfer associated with a compression wave,

By force sensor a device is meant that is configured to receive a force, to measure an own property that changes due to the application of this force, and to produce a detection signal responsive to this property. In particular, the property can be selected among the resistance of a conductive member, the capacity of a capacitor, a displacement, a temperature, a pressure.

The support on which the transducers of the tactile stimulation unit are arranged can be integrated or integral to the sensor interface. For instance, the device can comprise a prosthetic limb, such as a prosthetic forearm, or a prosthetic limb portion, such as a prosthetic finger or finger phalanx. Both the sensor and the transducer, or a plurality of these components, are arranged on this prosthesis, and the transducer is arranged to come into contact with an end portion of a user's stump. The stump can be, for example, a forearm or a proximal phalanx of a finger. This way, the stimulations can be transferred to the end portion of the stump, provided this is sensorially sound.

As an alternative, the support can be a component disconnected from the sensor interface, and configured to be worn on a body part that can even be different, or in any case far from the prosthetic body part or from the sensorially deficient natural body part. For example, the support can be a belt that can be conventionally tightened about a user's limb, such as an elastic bracelet.

The program means of the control unit can comprise a logical circuit configured to generate the control signal for the transducer responsive to the contact signal received from the sensor. As an alternative, the program means can comprise a microprocessor and a software resident in the control unit that are cooperatively configured to generate the control signal for the transducer responsive to the contact signal received from the sensor.

Advantageously, the program means is also configured to cause the control unit to produce the control signal when the contact signal carries out the second crossing, in a direction opposite to the first crossing, of the second contact threshold value, thus causing the tactile stimulation unit to transfer a further tactile stimulation to the healthy body part. This way, the user receives a tactile stimulation also when a complementary contact event takes place that is complementary of the event corresponding to the first crossing of the threshold, i.e. when an contact end or a contact beginning occurs, respectively.

According to alternative exemplary embodiments, the further tactile stimulation is the same as or different from the previous tactile stimulation. For instance, the two tactile stimulations can comprise respective vibrations that perceptibly differs from each other by at least one feature selected among its intensity, duration and frequency.

In particular the second contact threshold value is substantially coincident with the first contact threshold value.

In an exemplary embodiment, the tactile stimulation is a main tactile stimulation, and the tactile stimulation unit is configured to generate an auxiliary tactile stimulation different from the main tactile stimulation. The program means is configured to actuate the transducer so as to cause the tactile stimulation unit to transfer the auxiliary tactile stimulation to the above described sensorially sound user's body part, during said intermediate time. This provides the user, between the beginning and the end of the contact, for example, a reminder stimulation indicating the progression of the contact event.

For instance, the above indicated transducer is a main transducer, and the tactile stimulation unit comprises an auxiliary transducer arranged at a different position of the support with respect to said transducer, and is configured to generate the auxiliary tactile stimulation through the auxiliary transducer. This way, the auxiliary tactile stimulation is felt by the user at a point of the healthy body part different from the point where the main tactile stimulation is felt, so that the user can easily distinguish the main tactile stimulation from the auxiliary one.

As an alternative, or in addition, the tactile stimulation unit is configured to generate the main and the auxiliary tactile stimulations with at least one different perception feature. The main and the auxiliary tactile stimulations can be generated through the same transducer, or through distinct transducers arranged at different positions on the support. For instance, the main and the auxiliary tactile stimulations can comprise respective vibrations that are that perceptibly differs from each other by at least one feature. As an alternative, the first and the second stimulations can comprise perceptibly different vibrations sequences.

In a device according to the invention, the sensor interface can comprise at least one couple of contact sensors configured to be worn on opposable members of the user's prosthetic or sensorially deficient body part. This way, the tactile stimulation unit can be actuated when an event of gripping/releasing the object is performed by the user's prosthetic or sensorially deficient body part. In other words, the device is adapted to provide the user with a tactile stimulation corresponding to a condition in which the opposable members grip an object, and which preferably relates to a succession of displacements of this object retained between the opposable members.

In an exemplary embodiment, the contact sensors are force sensors, and the first and the second contact threshold value are grip force thresholds higher than a predetermined grip force limit value, such that the tactile stimulation unit transfers a steady-gripping stimulation on the object. Therefore, in the case of a rigid displacement of these opposable members, the object makes a displacement that is integral to the opposable members.

The device can comprise a couple of transducers each functionally associated with a respective contact sensor of the couple of contact sensors. Advantageously, the program means of the control unit is configured to emit respective control signals for the transducers when the signals coming from the couple of sensors exceed the respective contact threshold values, in order to actuate both the transducers of the couple. This way, the recognition of the grip condition is simplified, by the recognizing grip start and preferably of end grip end events at two different points of the sensorially sound body part.

In particular, in the device that is associated with the opposable members:

the sensor interface comprises a slide motion sensor arranged on at least one of the user's opposable members and configured to generate a slide/no slide movement signal of the opposable members and of the object with respect to each other;

the tactile stimulation unit comprises a further transducer, and is configured to generate, through the further transducer, a slide/no-slide movement tactile stimulation that can be haptically perceived by the user and that is responsive to the slide/no slide signal.

This way, a stimulation corresponding to a condition in which the object carries out a displacement integral with the opposable members can be transferred to the sensorially sound body part of the patient's body, during a displacement of the opposable members. This allows distinguishing a condition in which the grip force is strong enough to move the object integrally with the opposable members from a condition in which a relative slide movement occurs between the members and the object, so that the user can increase the grip force if he/she wishes to move the object integrally with the opposable members.

The slide motion sensor can be conventionally manufactured, and/or it can be made in one of the ways indicated in the description of the preferred exemplary embodiment.

Advantageously, the device comprises a displacement sensor configured to generate a displacement signal of the prosthetic or sensorially deficient body part, responsive to a displacement variable of the prosthetic or sensorially deficient body part, wherein the program means is configured to cause the control unit to produce a further control signal responsive to the displacement signal, such that the transducer provides a tactile displacement stimulation to the user when the displacement signal carries out a crossing of a displacement threshold value corresponding to said displacement. This way, the transducer can be actuated at the beginning/ at the end of a displacement of an object by the user's prosthetic or sensorially deficient body part. This allows the user to recognize also conditions of beginning and/or of end of a displacement of an object by a sensorially deficient or prosthetic limb.

The displacement sensor can be an accelerometer, to generate an acceleration signal of the user's prosthetic or sensorially deficient body part.

The contact signal, and/or the slide signal and/or the displacement signal being a reference signal, the program means can be configured to:

update a status of the reference signal with respect to a respective threshold value, when the reference signal carries out a crossing of the respective threshold value,

store the status of the reference signal into the control unit;

at each step of cyclically comparing the reference signal, compare a status in a current cycle of the reference signal, with respect to the respective threshold value, and a status in a cycle before the current status.

The program means of the control unit can be configured to sequentially actuate a plurality of transducers arranged at respective positions of the support when an event occurs of:

upwards crossing the contact threshold value by the contact signal, which passes from a first value lower than the contact threshold value to a second value higher than the contact threshold value;

upwards crossing the displacement threshold value by the displacement signal, which passes from a first value lower than the displacement threshold value to a second value higher than the displacement threshold value; or vice-versa;

downwards crossing the displacement threshold value by the displacement signal, wherein the displacement signal passes from a third value higher than the displacement threshold value to a fourth value lower than the displacement threshold value;

downwards crossing the contact threshold value by the contact signal, wherein the contact signal passes from a third value higher than the contact threshold value to a fourth value lower than the contact threshold value;

so that a specific combination of actuated transducers corresponds to each event. This way, the amputees or the sensorially deficient user can easily recognize a succession or manipulation tasks comprising the events of:

beginning of a step of contact with the object, i.e. end of a step of approaching the prosthetic or sensorially deficient body part to the object;

beginning of a step of moving the object by the prosthetic or sensorially deficient body part;

end of the step of moving the object;

end of the step of contact, i.e. beginning a step of releasing the object by the prosthetic or sensorially deficient body part.

A device according to claim 1, wherein the prosthetic or sensorially deficient body part is a portion of the user's natural lower limb or prosthetic lower limb, comprising a natural or prosthetic foot, and the sensor interface comprises a heel contact sensor and a toe side contact sensor configured to be arranged under the heel and under the front part, respectively, of a sole portion of the natural or prosthetic foot, and to generate a heel contact signal of and a toe side contact signal, respectively;

wherein the program means is configured to sequentially actuate a plurality of transducers, which are arranged at respective predetermined positions of the support, when events sequentially occur of:

upwards crossing a heel contact threshold value, corresponding to a contact condition between the heel and the ground, by the heel contact signal, which passes from a first value lower than the heel contact threshold value to a second value higher than the heel contact threshold value;

upwards crossing a toe side contact threshold value, corresponding to a contact condition between the front part of the sole portion and the ground, by the contact toe side signal, which passes from a first value lower than the toe side contact threshold value to a second value higher than the toe side contact threshold value;

downwards crossing the heel contact threshold value by the heel contact signal, wherein the heel contact signal passes from a third value higher than the heel contact threshold value to a fourth value lower than the heel contact threshold value;

downwards crossing the toe side contact threshold value by the toe side contact signal, wherein the toe side contact signal passes from a third value higher than the toe side contact threshold value to a fourth value lower than the toe side contact threshold value;

so that a specific combination of actuated transducers corresponds to each event. This way, the user can recognize a succession of events of:

beginning of a step of partial contact of the foot with the ground;

beginning of a step of complete contact of the foot with the ground, and of response to the load;

end of a step of complete contact of the foot with the ground, in particular the end of a support step in which one foot only is in contact with the ground;

end of a step of partial contact of the foot with the ground.

The sensor interface for an upper limb can have, for instance, the shape of a sensor glove or of a sensor thimble, which can be easily put on and worn. For a lower limbs, the sensor interface can have, for example, the shape of a sensor footwear or of a sensor insole. These exemplary embodiments are easy to be put on and worn.

The wearable support preferably comprises a sleeve arranged to connect the sensor interface with a part of a user's sensorially sound limb. This solution is preferable in the case of a device that has a removable sensor interface, such as a glove, a plurality of thimbles or a shoe. As an alternative, the wearable support can be integrated in a prosthetic limb, at a recess configured to house an amputee user's stump. This solution allows arranging the transducers on skin regions where a phantom limb sensation occurs, which correspond to the members that are to be artificially made sensitive by the device according to the invention,

Advantageously, the wearable support comprises a rechargeable battery for electrically supplying the control unit, the sensors and the transducers. Preferably a USB interface is provided to simplify recharging the battery.

### BRIEF DESCRIPTION OF THE DRAWINGS

The invention will be now shown with the following description of its exemplary embodiments, exemplifying but not limitative, with reference to the attached drawings, in which:

FIG. 1 diagrammatically shows a haptic feedback device according to the invention;

FIG. 2 is a block diagram that shows the operation of the program means of the control unit of a device according to the invention;

FIGS. 3 and 4 diagrammatically show devices according to further exemplary embodiments of the invention, comprising a plurality of sensor thimbles for a natural or prosthetic hand and a support wearable on a user's arm or forearm;

FIG. 5 diagrammatically shows a device according to the exemplary embodiment of FIG. 4, worn on a natural or prosthetic arm, in a step of gripping an object;

FIG. 6 diagrammatically shows a device according to an exemplary embodiment of the invention, incorporated in a prosthetic arm;

FIG. 7 is a block diagram illustrating the operation of the program means according to an exemplary embodiment of the invention;

FIG. 8 diagrammatically shows a device according to an exemplary embodiment of the invention, incorporated in a prosthetic finger or in a prosthetic phalanx;

FIG. 9 diagrammatically shows a device according to an exemplary embodiment of the invention, incorporated in a prosthetic leg;

FIG. 10 diagrammatically shows a device according to an exemplary embodiment, for a natural or prosthetic leg, in a step of support of contact of the whole foot sole;

FIG. 11 shows a sensor footwear for a sensor interface of a device according to an exemplary embodiment of the invention;

FIG. 12 shows a sensor insole for a sensor interface of a device according to an exemplary embodiment of the invention.

### DETAILED DESCRIPTION OF THE INVENTION

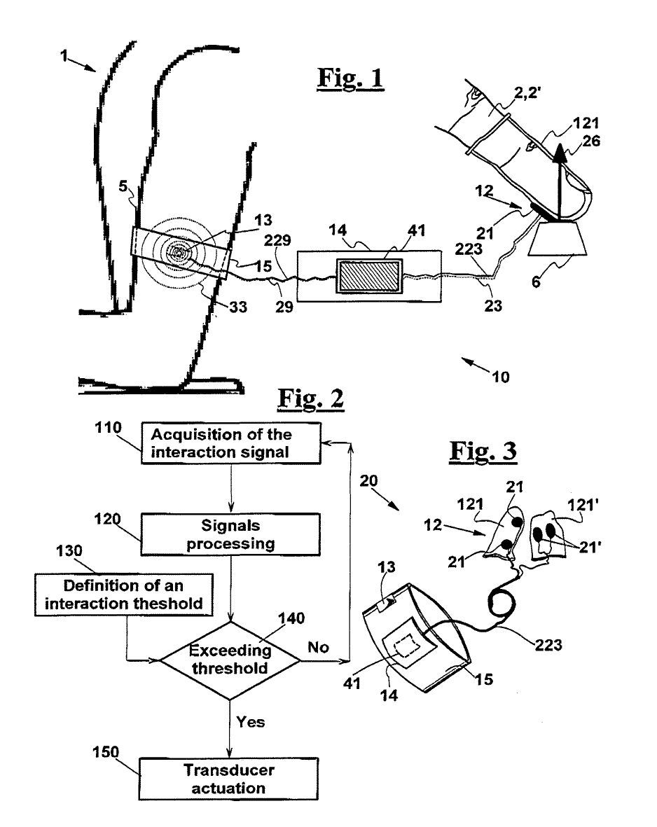

With reference to FIG. 1, a haptic feedback device 10 is described, according to the invention, for a user 1. The device is adapted to a user of a prosthetic body part 2, in this case a prosthetic portion of an upper limb such as a prosthetic finger, or for a user who is sensorially deficient at such a body part 2′, for example due to a spinal lesion or an ictus.

Device 10 comprises a sensor interface 12 equipped with at least one contact sensor 21. In this case, sensor interface 12 comprises a thimble 121 having at least one contact sensor 21 that is able to generate a contact signal 23, typically an electric signal, when sensor 21 receives an input 26 from an object 6, due to a contact or to a pressure that user 1 applies on object 6. For example, according to the sensor 21 type, input 26 can be a heat flux or an energy flux associated with an electromagnetic field or with a compression wave.

Sensor thimble 121 is worn on finger 2,2′, which can be a finger 2 of a prosthetic hand or a user's 1 sensorially deficient natural finger 2′.

In the representation of FIG. 1, object 6 is a button, for example a switch button or a key of a computer or typewriter keyboard. In this case, the sensor is arranged in a zone of finger 2,2′ that receive a force due to a stroke on key 6.

Sensor thimble 121 has further sensors, not shown, to provide contact signals 23 also in case of manipulations different from a stroke on key 6. For instance, sensors can be provided on the side surfaces of prosthetic or natural finger 2,2′, in order to provide a haptic feedback when a knob, not shown, is rotated.

Device 10 also comprises a tactile stimulation unit equipped with at least one transducer 13 configured to generate a tactile stimulation 33, adapted to be perceived by user 1. Transducer 13 can be of a predetermined type configured to generate a tactile stimulation 33 that can be perceived by the user. In particular, transducer 13 can be configured to generate a mechanical stimulation 33 that comprises a vibration having an intensity, a duration and a predetermined frequency. For example, transducer 13 can be of the type currently used for providing common mobile phones with a vibration mode.

In other exemplary embodiments, transducer 13 can be configured to provide a different tactile stimulation 33, for example an electric stimulation or a thermal stimulation.

In the exemplary embodiment of FIG. 1, transducer 13 is connected to a support 15 that can be worn by a patient. A wearable support can be, for instance, a sleeve such as a belt or a bracelet 15 that can be arranged and anatomically tightened with conventional means about a sensorially sound part 5 of user's body 1, for example about a forearm 5, as shown in FIG. 1. Transducer 13 is connected to wearable support 15, so as to transfer tactile stimulation 33 to sensorially sound body part 5, once support 15 has been put on.

With reference to FIGS. 1 and 2, device 10 also comprises a control unit 14 configured to carry out a step of acquisition 110 and a step of control 120 of contact signal 23 produced by sensor 21, to make a control signal 29 responsive to contact signal 23, and to transfer control signal 29 to transducer 13, in order to cause a step of actuation 150 of transducer 13, so that transducer 13 generates tactile stimulation 33 whose intensity and/or duration and/or frequency and/or another feature depends upon contact signal 23, as this is processed in control unit 14. More in detail, control unit 14 comprises a program means 41, which can be resident in a processor, that is configured to cause control unit 14 to produce control signal 29 responsive to contact signal 23. According to the invention, program means 41 is configured to cyclically carry out a step of checking 140 whether contact signal 23 has crossed a predetermined contact threshold value, which is the result of a previous step of definition 130, corresponding to a limit value of an interaction parameter 26 of an interaction between sensor 21 and object 6, which indicates a contact condition occurring between sensor 21 and object 6, for example, a predetermined force value. Program means 41 is also configured to cause transducer 13 to emit control signal 29, and then to generate stimulation 33, when contact signal 23 crosses the contact threshold value, passing from a value lower to a value higher than this threshold, or vice-versa. For instance, the contact threshold value can be the minimum force that must be applied on key 6 of a device as keyboard of an equipment, not shown, to generate an input for the equipment.

Even according to the invention, control unit 14 is configured for leaving transducer 13 substantially still during the intermediate time between a crossing of the contact threshold value and a subsequent crossing of the same contact threshold value, in a direction that is opposite to the previous crossing, or a following crossing of a distinct contact threshold value, still in a direction that is opposite to the previous crossing,

This way, transducer 13 can be actuated if an event occurs of beginning of a contact of user's 1 body part 2, in this case a finger, with an object such as key 6.

Device 10 comprises a first connection means 223 for transferring contact signal 23 from sensor interface 12 to control unit 14, and second connection means 229 for transferring control signal 29 from control unit 14 to the tactile stimulation unit, i.e. to at least one transducer 13. Even if in FIG. 1 shows a wired connection means 223,229, a different connection means can be provided, for instance a wireless means, as exemplified thereinafter.

Program means 41 can also be configured to actuate transducer 13 when contact signal 23 carries out the second crossing, so that the tactile stimulation unit transfers a further tactile stimulation 33 to sensorially sound body part 5 through transducer 13, wherein this tactile stimulation can be the same or different from the previous stimulation.

In the case of a key 6 of a keyboard, stimulations 33 relating to the beginning and to the end of the contact will be perceived as a single stimulation by user 1, since the contact with key 6 occurs in a very short time.

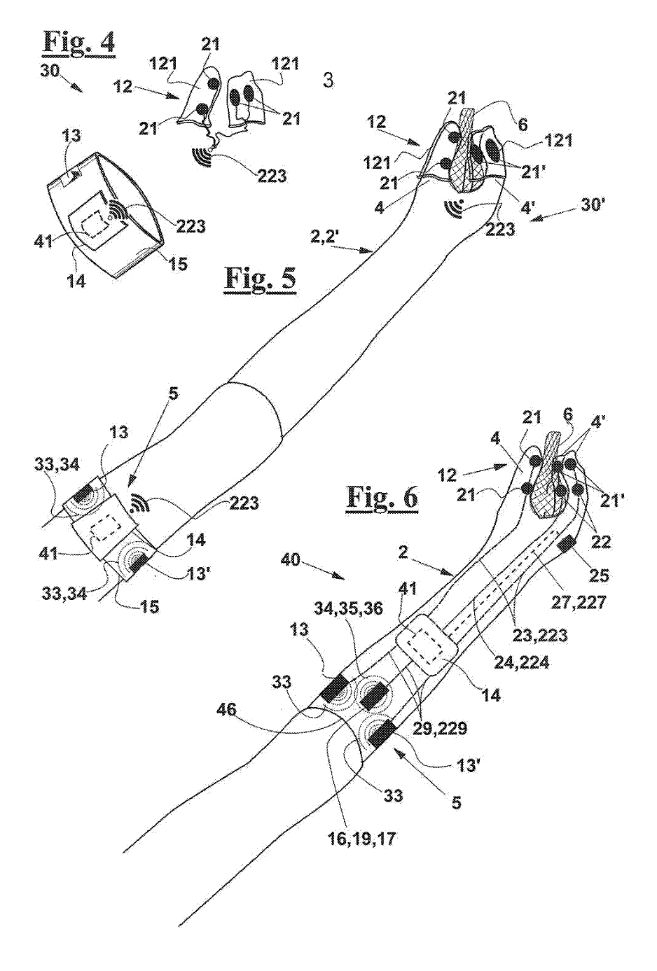

FIGS. 3 and 4 show haptic feedback devices 20,30 according to further advantageous exemplary embodiments of the invention, for the fingers of a hand, in which sensor interface 12 comprises at least one couple of contact sensors 21,21′ configured to be worn on opposable members 4,4′ of a user's prosthetic or sensorially deficient natural body part 2,2′, as shown in FIG. 5 with reference to a device 30′, which is a modification of device 30.

For instance, devices 20,30 comprise a plurality of thimbles 121 configured to be worn on opposable fingers 4,4′ of natural hand 2′ or of prosthetic hand 2, such as thumb 4 and forefinger and/or another finger 4′. A thimble 121′ can be configured to be worn on two or more fingers 4′ adjacent to each other and all opposable to thumb 4 of the user's hand.

As shown in FIG. 5, sensor interface 12 of devices 20,30,30′ can be worn both on fingers 4,4′ of a prosthetic arm 2, and on fingers 4,4′ of a natural arm 2′, that has a sensorially deficient portion, in particular fingers 4,4′.

Like device 10 of FIG. 1, devices 20,30,30′ comprise a support 15 that can be worn by a patient, on which the tactile stimulation unit having at least one transducer 13 is arranged. In devices 20,30,30′ control unit 14 can be advantageously integrated with wearable support 15.

Devices 30,30′ differ from device 20 in that they have wireless connection means 223, typically a Blueetooth type connection means, for transferring contact signal 23 from sensors 21,21′ to control unit 14, instead of wired connection means, like in device 20.

In the configuration of FIG. 5, if a minimum threshold compatible with a firm grip of object 6 is chosen as the contact threshold value, the transducer(s) of the tactile stimulation unit can be actuated if an event occurs in which opposable fingers 4,4′ grip object 6, in order to provide user 1 with a feedback of such grip event.

In particular, contact sensors 21,21′ can be force sensors, and the threshold value is chosen as a value not lower than a predetermined grip force value, so that stimulation 33 transferred by transducer 13 or by at least one of transducers 13,13′ corresponds to a grip on object 6 firm enough to allow it a displacement integral with opposable fingers 4,4′, when these are moved, in particular upwards, without sliding with respect to them.

Device 30′ differs from devices 20 and 30 in that the tactile stimulation unit comprises a plurality of transducers, in particular two transducers 13,13′. In a advantageous exemplary embodiment, each transducer 13,13′ is functionally associated with a respective contact sensor 21,21′. In other words, control unit 14 is preferably configured to actuate transducer 13 or transducer 13′ when a crossing occurs of a threshold force value by a signal coming from sensor 21 or from sensor 21′, respectively. For instance, transducer 13 is associated with sensor 21 of thumb finger 4, while transducer 13′ is associated with sensor 21′ of finger 4′ opposable to thumb 4. Advantageously, control unit 14 is configured to actuate both transducers 13,13′ when a crossing occurs of the threshold force value corresponding to predetermined the grip force, so that grip stimulation 33 can be easily distinguished by the other contact stimulations.

In another exemplary embodiment, not shown, sensor interface 12 can comprise a sensor glove, not shown, in which sensors are arranged at each finger that are electrically connected to a signal collection unit including a signal transmission unit, and/or a connector for connecting this signal collection unit with control unit 14 of the device.

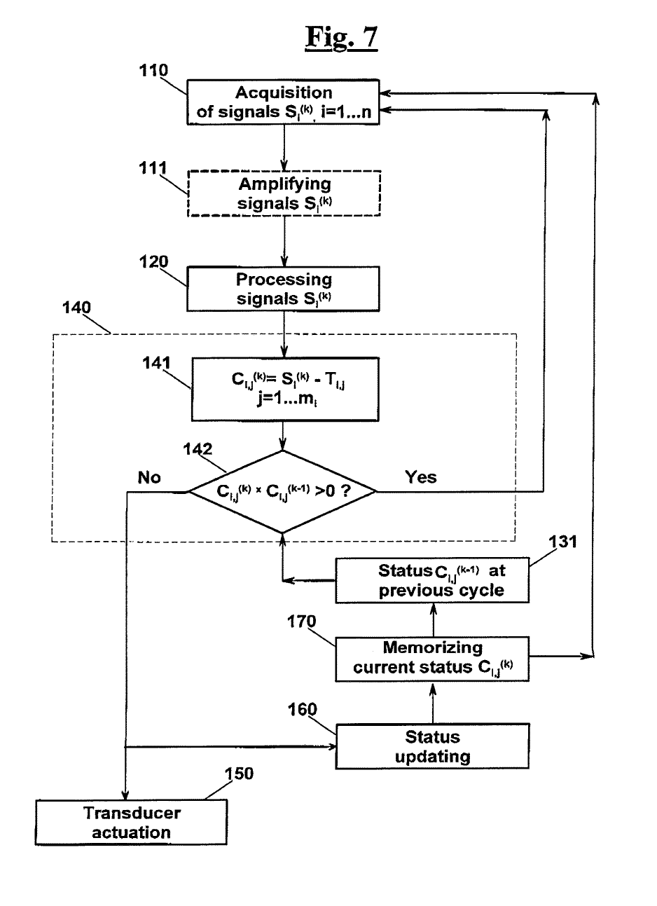

With reference to FIG. 6, a device 40 is described, according to an exemplary embodiment of the invention, that is incorporated in a prosthetic arm 2 for a amputee user, engaged with object 6 in a gripping condition. Device 40 can comprise two contact sensors for each finger 4,4′ of prosthesis 2, even if sensors 21,21′,22 of only three fingers 4,4′ are shown in the picture.

In this case, sensors 21 and transducers 13,13′ can be connected to control unit 14 by wired electric connections 223,224,227, which also are incorporated in prosthesis 2. Even in this case, however, a wireless connection means can be provided, as shown in FIG. 5.

In particular, sensors 21,21′ of each finger 4,4′ can be functionally connected, through control unit 14, with a respective transducer 13,13′. For example, the sensors arranged at a same prosthetic finger 4,4′ can be distinguished from one another by a suitable communication protocol. Even in this case, however, a wireless connection means can be provided, as shown in FIG. 5.

Regardless of the above, device 40 can optionally comprise at least one slide movement sensor 22 on at least one finger 4 or 4′, in order to generate a slide/no slide movement signal 24 relating to a relative slide movement o fingers 4,4′ and object 6, which is transferred to control unit 14 through connection means 227. In this case, device 40 can comprise at least one further transducer 19 associated with slide movement sensor(s) 22, and program means 41 can be configured in such a way that further transducer 19 generates a slide/no-slide movement tactile stimulation 35 that can be perceived by user 1 and that is responsive to slide/no slide movement signal 24, which can be transferred to the sensorially sound part of the patient's body.

For instance, slide movement sensor 22 can comprise a sensor of mechanical vibrations. In fact, the relative slide movement of fingers 4,4′ and object 6 is normally associated with mechanical vibrations that firstly depend on the surface roughness of object 6.

As an alternative, slide movement sensor 22 can comprise a sensor configured to measure a heat exchange condition, like a temperature sensor. In fact, the relative slide movement between fingers 4,4′ and object 6 is normally associated with a frictional dissipation of mechanical energy, which generates heat.

As an alternative, slide movement sensor 22 can comprise a force sensor configured to measure a force component differently oriented with respect to the component normal to the surface of finger 4′ at sensor 22.

As an alternative, slide movement sensor 22 can comprise a conventional distance sensor configured to measure a distance from a reference point fixedly arranged on object 6.

Regardless of the above, device 40 can comprise at least one displacement sensor or inertial sensor 25 configured to generate a displacement signal 27 of prosthetic body part 2, responsive to an own displacement variable such as the speed or the acceleration. In the latter case, displacement sensor 25 can be an accelerometer. In an exemplary embodiment of FIG. 6, displacement sensor 25 can be arranged at a position corresponding to the back of a hand, in order to detect status of beginning/end of a displacement of the whole hand.

In this case, control unit 14 is also configured to receive and analyze displacement signal 27 through connection means 227, to compare it with a threshold value of the same displacement variable, and to produce a control signal 29, responsive to displacement signal 27, in order to actuate a respective transducer 13,13′ or 16, according to the same procedure as above, comprising steps 110-150 (FIG. 2), by which contact signal 23 is treated.

More in detail, program means 41 is configured for cyclically comparing displacement signal 27 with a predetermined displacement threshold value, corresponding for instance to a speed limit value and/or to an acceleration limit value during the displacement of hand or of arm 2, and for producing control signal 29 and transferring it to transducer 13,13′ or 16 so that the latter generates the displacement stimulation 33 or 36, respectively, when displacement signal 27 crosses this displacement threshold value, passing from a value lower to a value higher than the threshold, or vice-versa.

This way, the user can be provided with a haptic feedback at the beginning/at the end end of a displacement of a limb, in particular, along with a displacement of an object 6.

Transducers 13,13′,16,19 are advantageously arranged in a recess 46 of prosthetic limb 2, preferably they are arranged in such a way to come into contact with a user's skin zone where a phantom limb sensations occur.

Even if slide movement sensor 22 and of displacement movement sensor 25 are shown only in device 40, which is incorporated in prosthesis 2, they can also be provided in such devices as devices 30 and 30′, which comprise parts to be worn on prosthetic or natural limb 2,2′, along with the respective communication means and possible transducers 16,19 distinct from transducers 13,13′.

Sill with reference to FIGS. 5 and 6, the tactile stimulation unit of devices 30′ and 40 can be configured to generate an auxiliary tactile stimulation 34 distinct from main tactile stimulation 33, in particular at regular time intervals, during the intermediate time between a crossing of the contact threshold value, by contact signal 23, and a subsequent crossing, in an opposite direction, of the same contact threshold value or of another contact threshold value. In this case, program means 41 is configured to cause control unit 14 to produce a control signal 29, preferably every predetermined interval of time. This way, transducer 13,13′, or a different transducer 17, transfers auxiliary tactile stimulation 34 to sensorially sound body part 5 during the intermediate time.

In particular, as shown in FIG. 5, the tactile stimulation unit can be configured to generate auxiliary tactile stimulation 34 through transducer(s) 13,13′ that generate main tactile stimulation 33, wherein the auxiliary tactile stimulation perceptibly differs from main tactile stimulation 33 by a feature. In the case of stimulations comprising a vibration, this feature can be, for instance, the duration, the intensity or the frequency of the vibration. As an alternative, main and auxiliary tactile stimulations 33,34 can be heterogeneous tactile stimulations, each of them selected, for example, among electric stimulations, stimulations comprising a plurality of mechanical vibrations, thermal stimulations.

With reference to FIG. 6, the tactile stimulation unit comprises an auxiliary transducer 17 different from main transducer 13 and arranged at a different position of support 15, with respect to main transducer 13. In this case, the tactile stimulation unit is configured to generate auxiliary tactile stimulation 34 through auxiliary transducer 17.

Moreover, on a same finger 4 of a prosthesis (FIG. 6) or on a same wearable finger 4′ (FIG. 5) a plurality of contact sensors can be arranged ech corresponding to a different portion of the finger 4,4′, for example to different phalanxes.

As shown in FIG. 7, in an exemplary embodiment, program means 41 of control unit 14 are configured to carry out, in each operation cycle “k”, a step 110 of acquiring n numerical values Sᵢ^{(k)} i=1 . . . n of n contact signals 23, of slide movement signals 24 or of displacement signals 27 provided by contact sensors 21, by slide movement sensors 22 or by displacement sensors 25, respectively. If necessary, signals 23,24,27 may undergo to an amplification step 111, which is shown in dashed line because it is facultative. A step 120 follows of processing signals Sᵢ^{(k)}, which may comprise, for instance, a step of computing at least one derivative of at least one signal 23,24,27. Advantageously, the numerical values of the signals after steps 111 and 120 are in any case indicated as Sᵢ^{(k)} i=1 . . . n, regardless of the effect of these steps.

As already indicated while describing FIG. 2, a step 140 is carried out of checking whether value Sᵢ^{(k)} of each contact signal 23, slide movement signal 24 or displacement signal 27 has crossed, when passing from previous cycle (k−1) to current cycle (k), a predetermined contact or displacement threshold value T_{i,j}. For each signal 23,24,27, m threshold values j=1 . . . m, can be provided, each corresponding to a checked crossing. The crossing can take place upwards, in which case the signal passes from a previous value Sᵢ^{(k-1)} lower than threshold value T_{i,j} to a current value Sᵢ^{(k)} higher than threshold T_{i,j} or, vice-versa, it can take place downwards, in which case the signal passes from a previous value Sᵢ^{(k-1)} higher than threshold T_{i,j} to a current value Sᵢ^{(k)} lower than threshold T_{i,j}. Step of checking 140 comprises a step 141 of computing the sign of variable C_{i,j}^{(k)}=Sᵢ^{(k)}−T_{i,j}, which is positive if at k^{th} cycle signal Sᵢ^{(k)} is higher than threshold T_{i,j}, and is negative if at k^{th} cycle signal Sᵢ^{(k)} is lower than threshold T_{i,j}. In both cases, and for each value of signal Sᵢ^{(k)}, a step 142 follows of comparing value Sᵢ^{(k-1)} that i^{th} signal had in the previous cycle, after a step 131 of retrieving the value of Sᵢ^{(k-1)}, if C_{i,j}^{(k)}×C_{i,j}^{(k)}>0, with obvious meaning of the symbols, no crossing of threshold T_{i,j} has occurred between cycle (k−1) and cycle (k). In this case, program means 41 passes to subsequent cycle (k+1) carrying out the same check step. On the contrary, if C_{i,j}^{(k)}×C_{i,j}^{(k)}>0 is not true, a crossing of threshold T_{i,j} has occurred, and program means 41 proceed with step 150 of actuating transducer(s) 13,16,19 associated with i^{th} sensor 21,22 or 25, thus causing a tactile contact stimulation 33, or a tactile slide movement stimulation 35 or a tactile displacement stimulation 36, which can be a short sequence of vibrations, which is transferred to sensorially sound part 5 of the patient's body at a position thereof. Moreover, program means 41 carries out a step 160 of updating the status of each sensor 21,22,25 with respect to a respective threshold T_{i,j}, and a step 170 of memorizing the new value of variable C_{i,j}^{(k),} or only the sign of this variable, so that this is available for check step 140 in subsequent (k+1)^{th} cycle.

In an exemplary embodiment, with reference to FIGS. 5 and 6, a haptic feedback device has a sensor interface 12 comprising at least one contact sensor 21 and at least one displacement sensor 25, as shown only in FIG. 6, which are functionally associated to respective transducers 13 or 13 and 16 fixed on wearable support 15 or in recess 46 of prosthesis 2, in order to be arranged at predetermined positions on sensorially sound body part 5. Moreover, program means 41 is configured to sequentially actuate transducers 13 when an event occurs of:

upwards crossing the contact threshold value by contact signal 23 referred to the interaction between the prosthetic or sensorially deficient body part 2,2′ of user's body and object 6, i.e. a passage from a value lower to a value higher than the contact threshold value. This interaction can be a grip force, if the sensor is arranged on a member of a couple of at least two opposable members of prosthetic or sensorially deficient user's 1 body part 2,2′, such as natural or prosthetic fingers 4,4′, or if two sensors are arranged on respective members of a couple of such opposable members 4,4′. In this case, tactile contact stimulation 33 generated by transducer(s) 13 enables user 1 to recognize the beginning of a step of contact with object 6 by means of its own prosthetic or sensorially deficient body part 2,2′, or a gripping step by means of opposable members 4,4′ of prosthetic or sensorially deficient natural body part 2,2′;

upwards crossing the displacement threshold value by displacement signal 27 referred to the displacement of object 6 that user 1 causes through prosthetic or sensorially deficient body part 2,2′, i.e. a passage from a value lower to a value higher than the displacement threshold value. In particular, the displacement can be a lifting displacement. In this case, tactile displacement stimulation 33 generated by transducer(s) 13 enables user 1 to recognize the beginning a displacement step, in particular the beginning of a step of lifting object 6 by prosthetic or sensorially deficient body part 2,2′;

downwards crossing the displacement threshold value by displacement signal 27, i.e. a passage from a value lower to a value higher than the displacement threshold value. In particular, in this case, tactile stimulation 33 generated by transducer(s) 13 enables user 1 to recognize the end of a displacement step, in particular of a lifting displacement, of object 6 by prosthetic or sensorially deficient body part 2,2′;

downwards crossing the contact threshold value by contact signal 23, i.e. a pass from a value lower to a value higher than the contact threshold value. In the case of a step of gripping object 6, this downwards crossing relates to the release of object 6 by opposable members 4,4′ of prosthetic or sensorially deficient body part 2. In this case, tactile stimulation 33 generated by transducer(s) 13 enables user 1 to recognize the end of a step of contact, in particular of a step of gripping object 6 by its own body prosthetic or sensorially deficient natural body part 2,2′.

With reference to FIG. 8, a device is described 50 comprising or integrated in a prosthetic finger phalanx 18. Contact sensor 21 is arranged at a distal portion 18′ of prosthesis 18, while transducer 13 is arranged on a support 15 within a proximal recess portion 47 configured to receive an end portion 5 of a residual part or stump 5′ of user's finger 47′, which is sensorially sound. Transducer 13 is arranged to come into contact with end portion 5. In an exemplary embodiment, control unit 14 is incorporated in the body of prosthetic phalanx 18.

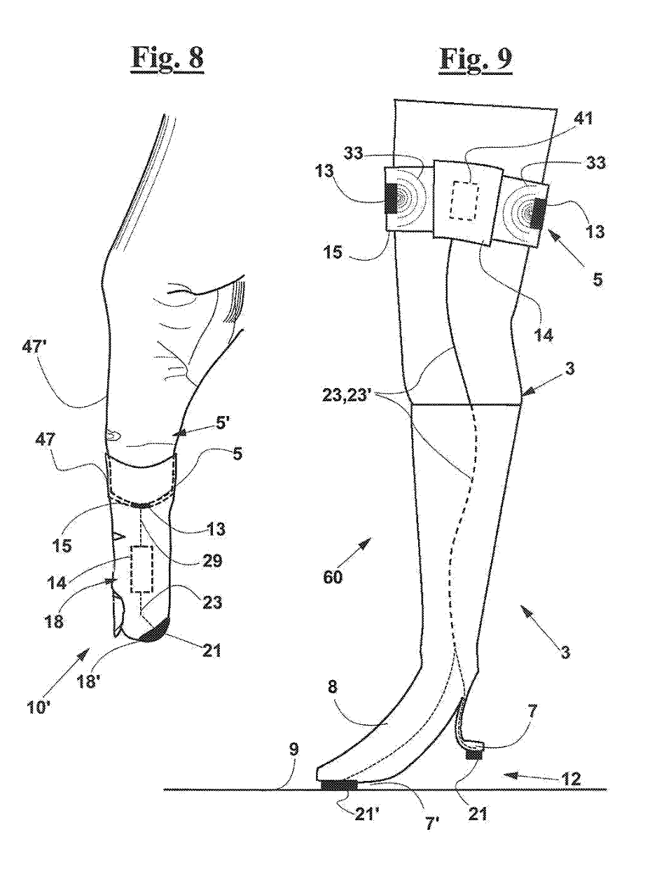

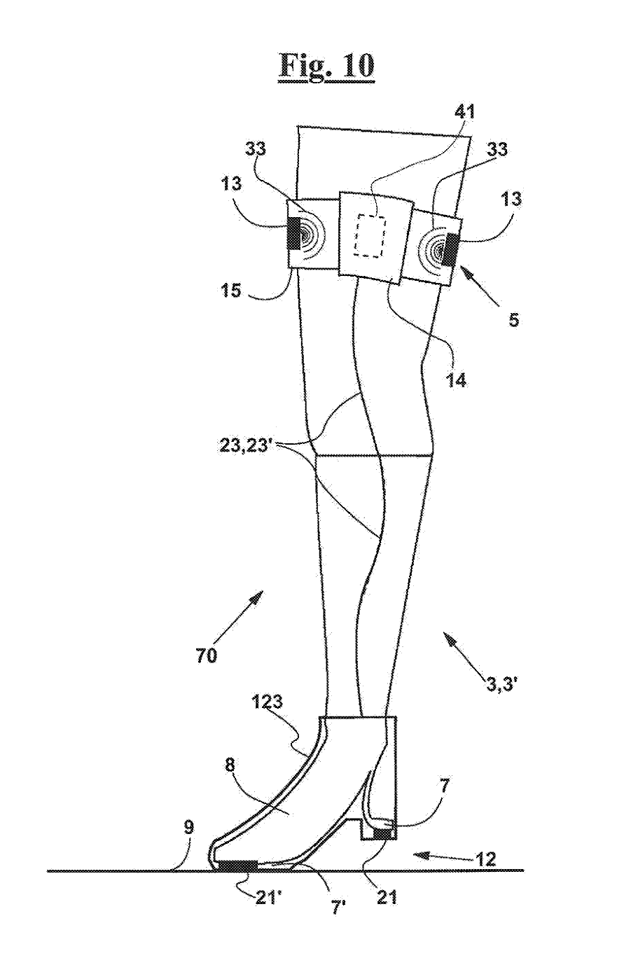

With reference to FIG. 9, a device 60 is described that is suitable for an amputee user missing a lower limb portion, in which the prosthetic or sensorially deficient natural body part is a portion of a prosthetic lower limb 3, comprising a prosthetic foot 8. Sensor interface 12 comprises at least one heel contact sensor 21 arranged at a prosthetic heel 7, and a toe side contact sensor 21′ arranged at front portion 7′ of the sole of prosthetic foot 8.

Device 60 comprises at least two transducers 13, respectively associated with heel sensor 21 and with toe side sensor 21′. Transducers 13 are connected to a wearable support 15, preferably a to sleeve or to a belt that can be anatomically arranged and fastened about a sensorially sound part 5 of user's body 1 by conventional means, for example, as shown in FIG. 9, about a thigh portion 5. According to an alternative exemplary embodiment, not shown, wearable support 15 can be an integrated wearable support, i.e. it can be integral to prosthesis 3. In particular, support 15 is configured to be arranged within a recess proximal portion configured to receive an end portion of a sensorially sound stump of a user's leg. Transducer 13 is arranged to come into contact with this end portion. In an exemplary embodiment, not shown, control unit 14 is incorporated within the body of prosthetic leg 3.

Transducer 13 is connected to wearable support 15, so as to transfer tactile stimulation 33 to sensorially sound body part 5, once support 15 has been put on.

With reference to FIG. 10, a haptic feedback device 70 is described that is suitable for an amputee user missing a lower limbs portion, or for a user who is sensorially deficient at foot 8, wherein the prosthetic or sensorially deficient body part is a prosthetic portion 3 or a natural portion 3′ of a lower limb, comprising foot 8. Device 70 differs from device 60 of FIG. 10 in that it comprises a shoe 123 that can be worn on natural or prosthetic foot 8, in which heel and toe-side contact sensors 21,21′ are incorporated. An exemplary embodiment of shoe 123 is shown and described with reference to FIG. 11.

Still with reference to devices 60 and 70 of FIGS. 9 and 10, program means 41 is configured for transferring a control signal 29 to transducers 13, which are therefore actuated, when contact signal 23,23′ from heel or toe side sensor 21,21′ exceeds a predetermined heel or toe side contact threshold value, for instance when heel contact signal 23 passes from a first value to a second value of the heel contact signal corresponding to values of force exchanged between the heel 7 or between the front portion 7′, respectively, and the ground 9, which are lower and higher than the predetermined force limit value. Similarly, program means 41 is configured to transfer a control signal 29 to transducers 13, which are therefore actuated, when heel or toe side force signal 23,23′ downwards crosses the respective heel and toe-side force threshold value, corresponding to heel and toe-side contact force limit values. The actuation of transducers 13 is preferably occurs in such a way that that a specific combination of actuated transducers 13 corresponds to each of said upwards and downwards crossing events, such that the user of device 60 or 70 can recognize mechanical events of
beginning of a step of partial contact of foot 8 with ground 9;
beginning of a step of complete contact of foot 8 with ground 9 and of response to the load;
end of a step of complete contact of foot 8 with ground 9, in particular the end of a support step in which foot 8 only is in contact with ground 9;
end of a step of partial contact of foot 8 with ground 9.

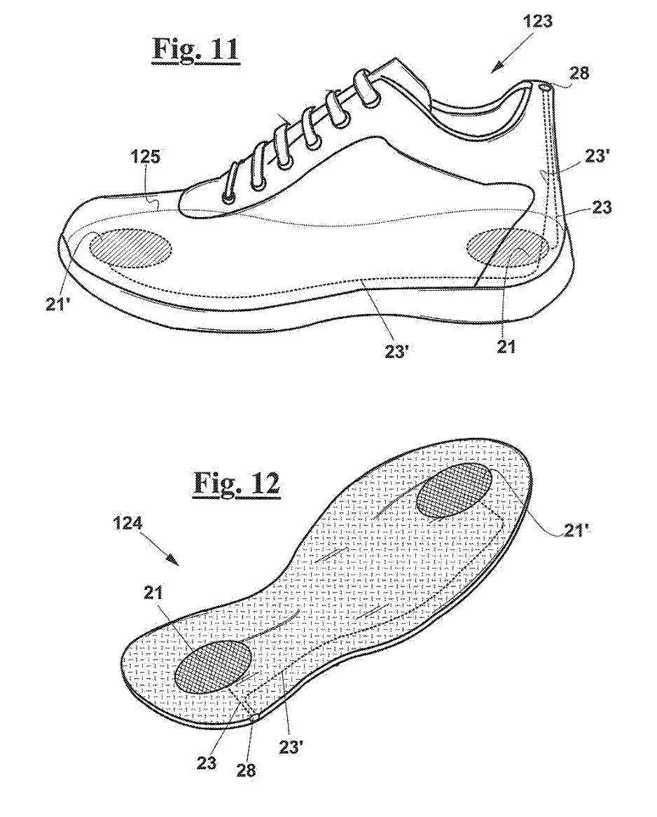

With reference to FIGS. 11 and 12, a haptic feedback device may comprise a sensor footwear 123 or an insole 124 for introduction into a shoe as the sensor interface. In a surface or inner layer of a sole 125 of shoe 123 or of insole 124 two heel and toe-side contact sensors 21,21′ are provided, which are electrically connected to a signal collection unit equipped with a connector 28 for connecting the collection unit with control unit 14 of the haptic feedback device. As an alternative, the signal collection unit can be equipped with a signal transmission unit, not shown, for the iteration signals.

Wearable support 15 of the type shown in FIGS. 3-6 and 8-10 can comprise a rechargeable battery, not shown, which provides energy to control unit 14, to sensors 21,21′,22,25 and to transducers 13,13′,16,19. In this case, the wired connections indicated for example by numbers 23,29 can comprise electric supply connections starting from the battery. A USB interface, not shown, can also be provided on wearable support 15 for recharging the battery.

In an exemplary embodiment, not shown, suitable for a user who is multiamputee and/or sensorially deficient in a plurality of parts of the body, for example in a plurality of limbs, a haptic feedback device comprises a plurality of sensor interfaces each configured to be arranged on a respective sensorially deficient or prosthetic body part, with respective contact sensors and possibly displacement and/or slide movement sensors. In this exemplary embodiment, each sensor interface preferably comprises a signal collection unit for collecting contact signals, displacement signals or sliding movement signals, and is equipped with a signal transmission unit, as shown in FIG. 4, for transferring these signals to the control unit.

The foregoing description exemplary embodiments of the invention will so fully reveal the invention according to the conceptual point of view, such that others, using the prior art, will be able to modify and/or adapt for various applications such embodiment without further research and without parting from the invention, and, accordingly, it is therefore to be understood that such adaptations and modifications will have to be considered as equivalent to the specific embodiments. The means and the materials to realize the different functions described herein could have a different nature without, for this reason, departing from the field of the invention. Is meant that the phraseology or terminology that is employed herein is for the purpose of description and not of limitation.

1. A haptic feedback device (10,20,30,30′,40,50,60,70) for a user's (1) prosthetic body part (2,3) or sensorially deficient body part (2′,3′), said device comprising:
a sensor interface (12) configured to be arranged on a prosthetic body part (2,3) or sensorially deficient body part (2′,3′),
wherein said sensor interface (12) comprises a contact sensor (21,21′) configured to generate a contact signal (23,23′) according to a contact condition occurring between said sensor and an object (6) of the outside of said user's body (1),
a tactile stimulation unit comprising a transducer (13,13′) configured to generate a tactile stimulation (33) adapted to be perceived by a healthy body part (5) of said user (1);
a support (15) configured to be worn on a region (5) of said user's body (1), wherein said tactile stimulation unit is arranged on said support (15) so as to transfer said tactile stimulation to said healthy body part (5);
a control unit (14) configured to receive said contact signal (23,23′) from said contact sensor (21,21′) and to produce a control signal (29) for actuating the transducer, wherein said control unit comprises a program means (41) configured to cause said control unit (14) to produce said control signal (29) responsive to said contact signal (23), such that said transducer (13,13′) provides said tactile stimulation (33) to said healthy body part (5) of said user (1);
characterized in that said program means (41) is also configured to carry out the steps of:
cyclically comparing said contact signal (23,23′) and a first contact threshold value (T_{i,j}) corresponding to said contact condition;
producing said control signal (29) and actuating said transducer (13,13′) when said contact signal (23,23′) carries out a first crossing of said first contact threshold value (T_{i,j}), such that said tactile stimulation unit transfers said tactile stimulation (33) to said healthy body part (5);
leaving said transducer (13,13′) substantially still in an intermediate time between said first crossing of said first contact threshold value (T_{i,j}) and a second crossing of a second contact threshold value (T_{i,j}) by said contact signal (23,23′), in a direction opposite to said first crossing,
so that said transducer (13,13′) is actuated at the beginning and/or at the end of a contact between said prosthetic body part (2,3) or sensorially deficient body part (2′,3′) and said object (6).

2. A device according to claim 1, wherein said program means (41) is also configured to cause said control unit (14) to produce said control signal (29) when said contact signal (23,23′) carries out said second crossing, in a direction opposite to said first crossing, of said second contact threshold value (T_{i,j}), thus causing said tactile stimulation unit to transfer a further tactile stimulation (33) to said healthy body part (5), wherein said further tactile stimulation is selected from the group consisting of:
a further tactile stimulation (33) equal to said tactile stimulation;
a further tactile stimulation (33) different from said tactile stimulation.

3. A device according to claim 2, wherein said second contact threshold value (T_{i,j}) is substantially coincident with said first contact threshold value (T_{i,j}).

4. A device according to claim 1, wherein said tactile stimulation is a main tactile stimulation (33), wherein said tactile stimulation unit is configured to generate an auxiliary tactile stimulation (34) different from said main tactile stimulation (33), and said program means (41) is configured to actuate said transducer (13,13′,17) so as to cause said tactile stimulation unit to transfer said auxiliary tactile stimulation to said healthy body part (5), which is sensorially sound, during said intermediate time,
wherein said tactile stimulation unit is selected from the group consisting of:
a tactile stimulation unit comprising said transducer, i.e. a main transducer (13,13′), and an auxiliary transducer (17) arranged at a different position of said support (15) with respect to said main transducer (13,13′), and configured to generate said auxiliary tactile stimulation (34) through said auxiliary transducer (17), such that said auxiliary tactile stimulation (34) is felt by the user (1) at a point of said healthy body part (5) different from the point where said main tactile stimulation (33) is felt;
a tactile stimulation unit configured to generate said main and auxiliary tactile stimulations (33,34) with at least one perception feature different from one another.

5. A device according to claim 1, wherein said sensor interface (12) comprises at least one couple of contact sensors (21,21′) configured to be worn on opposable members (4,4′) of said prosthetic or sensorially deficient body part (2,2′,3,3′), such that said tactile stimulation unit is actuated in the case of a gripping/releasing event of said object (6) by said user's (1) prosthetic or sensorially deficient body part (2,2′,3,3′),

6. A device according to claim 5, wherein said contact sensors of said couple of contact sensors are force sensors (21,21′), and said first and second contact threshold values (T_{i,j}) are grip force thresholds higher than a predetermined grip force limit value, such that said tactile stimulation unit transfers a steady-gripping stimulation (33) on said object (6).

7. A device according to claim 5, wherein said sensor interface (12) comprises a slide motion sensor (22) arranged on at least one of said user's (1) opposable members (4,4′) and configured to generate a slide/no slide movement signal (24) of said opposable members (4,4′) and said object (6), and
said tactile stimulation unit comprises a further transducer (19), and is configured to generate, through said further transducer (19), a slide/no-slide movement tactile stimulation (35) that can be haptically perceived by said user (1) and that is responsive to said slide/no slide movement signal (24).

8. A device according to claim 1, comprising a displacement sensor (25) configured to generate a displacement signal (27) of said prosthetic or sensorially deficient body part (2,2′,3,3′), said displacement signal (27) responsive to a displacement variable of said prosthetic or sensorially deficient body part (2,2′,3,3′), wherein said program means (41) is configured to cause said control unit (14) to produce a further control signal (29) responsive to said displacement signal (27), such that said transducer (13,13′,16) provides a tactile displacement stimulation (36) to said user (1) when said displacement signal (27) carries out a crossing of a displacement threshold value (T_{i,j}) corresponding to said displacement,
such that said respective transducer (13,13′,16) is actuated at the beginning/end of a displacement of said object (6) by said prosthetic or sensorially deficient body part (2,2′,3,3′).

9. A device according to claim 8, wherein said displacement sensor is an accelerometer (25) configured to generate an acceleration signal (27) of the user's prosthetic or sensorially deficient body part.

10. A device according to claim 1, wherein said contact signal, and/or said slide signal and/or said displacement signal (23,23′,24,27) is a reference signal, and said program means (41) is configured to:
update a status of said reference signal with respect to a respective threshold value (T_{i,j}), when said reference signal (23,23′,24,27) carries out a crossing of said respective threshold value (T_{i,j}),
store said status of said reference signal (23,23′,24,27) into said control unit (14);
at each step of cyclically comparing said reference signal (23,23′,24,27), compare a status in a current cycle of said reference signal (23,23′,24,27) with respect to said respective threshold value, and a status in a cycle before said current status.

11. A device according to claim 6, wherein said program means (41) is configured to sequentially actuate a plurality of transducers (13,13′,16), which are arranged at respective positions of said support (15), when an event occurs of:
upwards crossing said contact threshold value by said contact signal (23,23′), which passes from a first value lower than said contact threshold value to a second value higher than said contact threshold value;
upwards crossing said displacement threshold value by said displacement signal (27), which passes from a first value lower than said displacement threshold value to a second value higher than said displacement threshold value;
or vice-versa;
downwards crossing said displacement threshold value by said displacement signal (27), wherein the displacement signal (27) passes from a third value higher than said displacement threshold value to a fourth value lower than said displacement threshold value;
downwards crossing said contact threshold value by said contact signal, wherein the contact signal (23) passes from a third value higher than said contact threshold value to a fourth value lower than said contact threshold value;
or vice-versa, respectively;
in such a way that a specific combination of actuated transducers (13,13′,16) corresponds to each of said events, so that said user (1) is enabled to recognize mechanical events of:
beginning of a step of contact with said object (6), i.e. end of a step of approaching said prosthetic (2′) or deficient (2) body part to said object (6);
beginning of a step of moving said object (6) by said prosthetic body part (2) or sensorially deficient body part (2′);
end of said step of moving said object (6);
end of said step of contact, i.e. beginning a step of releasing said object (6) by said prosthetic body part (2) or sensorially deficient body part (2′).

12. A device according to claim 1, wherein said prosthetic or sensorially deficient body part is a portion of said user's (1) natural lower limb (3′) or prosthetic lower limb (3), comprising a natural or prosthetic foot (8), and said sensor interface (12) comprises a heel contact sensor (21) and a toe side contact sensor (21′) configured to be arranged under the heel (7) and under the front part (7′) of a sole portion of said natural or prosthetic foot (8), respectively, and configured to generate a heel contact signal (23) and a toe side contact signal (23′), respectively,
wherein said program means (41) is configured to sequentially actuate a plurality of transducers (13,13′), which are arranged at respective predetermined positions of said support, when events sequentially occur of:
upwards crossing a heel contact threshold value, corresponding to a contact condition between the heel (7) and the ground (9), by said heel contact signal (23), which passes from a first value lower than said heel contact threshold value to a second value higher than said heel contact threshold value;
upwards crossing a toe side contact threshold value, corresponding to a contact condition between the front part (7′) of the sole portion and the ground (9), by said toe side contact signal (23′), which passes from a first value lower than said toe side contact threshold value to a second value higher than said toe side contact threshold value;
downwards crossing said heel contact threshold value by said heel contact signal (23), wherein the heel contact signal (23) passes from a third value higher than said heel contact threshold value to a fourth value lower than said heel contact threshold value;
downwards crossing said toe side contact threshold value by said toe side contact signal (23′), wherein the toe side contact signal (23′) passes from a third value higher than said toe side contact threshold value to a fourth value lower than said toe side contact threshold value;
so that a specific combination of actuated transducers (13,13′) corresponds to each of said events.

13. A device according to claim 1, wherein said sensor interface (12) comprises an interface selected from the group consisting of:
a sensor glove;
at least one sensor thimble (121);
a sensor footwear (123);
a sensor insole (124).

14. A prosthetic limb (2,3) comprising a haptic feedback device (40,50,60) according to claim 1, wherein said haptic feedback device (40,50,60) is incorporated in said prosthetic limb.

15. The prosthetic limb (2,3) according to claim 14, wherein said support (15), which can be worn by a user, is integrated in an recess (46,47) of said prosthetic limb (2,3) configured for housing an amputee user's stump (46′,47′).

16. A device according to claim 5, wherein said contact signal, and/or said slide signal and/or said displacement signal (23,23′,24,27) is a reference signal, and said program means (41) is configured to:
update a status of said reference signal with respect to a respective threshold value (T_{i,j}), when said reference signal (23,23′,24,27) carries out a crossing of said respective threshold value (T_{i,j}),
store said status of said reference signal (23,23′,24,27) into said control unit (14);
at each step of cyclically comparing said reference signal (23,23′,24,27), compare a status in a current cycle of said reference signal (23,23′,24,27) with respect to said respective threshold value, and a status in a cycle before said current status.

17. A device according to claim 6, wherein said contact signal, and/or said slide signal and/or said displacement signal (23,23′,24,27) is a reference signal, and said program means (41) is configured to:
update a status of said reference signal with respect to a respective threshold value (T_{i,j}), when said reference signal (23,23′,24,27) carries out a crossing of said respective threshold value (T_{i,j}),
store said status of said reference signal (23,23′,24,27) into said control unit (14);
at each step of cyclically comparing said reference signal (23,23′,24,27), compare a status in a current cycle of said reference signal (23,23′,24,27) with respect to said respective threshold value, and a status in a cycle before said current status.
